(19) United States
(12) Patent Application Publication
(10) Pub. No.: US 2025/0194966 A1
(43) Pub. Date: Jun. 19, 2025

(54) REPOSITIONABLE SENSOR WITH BIASING MEMBER

(71) Applicant: Covidien LP, Mansfield, MA (US)

(72) Inventor: Abel Valdes, Lafayette, CO (US)

(21) Appl. No.: 18/977,313

(22) Filed: Dec. 11, 2024

Related U.S. Application Data
(60) Provisional application No. 63/610,384, filed on Dec. 14, 2023.

(51) Int. Cl. A61B 5/1455; A61B 5/00

(52) U.S. Cl. CPC A61B 5/14552; A61B 5/6832; A61B 2560/0462; A61B 2562/164; A61B 2562/185

(57) ABSTRACT

A medical sensor includes an outer cover that forms a pocket with an opening. The medical sensor also includes one or more electrical components positioned within the outer cover. The medical sensor further includes a biasing member coupled to the outer cover, wherein the biasing member adjusts from an initial shape to an adjusted shape to adjust a dimension of the opening to enable the opening to receive a portion of a patient.

### CROSS-REFERENCE TO RELATED APPLICATION

The present application claims the benefit of U.S. Provisional Patent Application Ser. No. 63/610,384, filed on Dec. 14, 2023, the entire content of which is incorporated herein by reference.

### FIELD

The present disclosure generally relates to medical monitoring devices, and more particularly to a sensor that includes a biasing member to facilitate repositioning of the sensor on a patient.

### BACKGROUND

Various medical monitoring devices may be used to monitor physiological characteristics of an individual. For example, various sensors may be used to measure temperature, pressure, oxygen, and other physiological characteristics of the individual. One such sensor, a pulse oximetry sensor, may be used to measure oxygen saturation levels in blood of the individual by utilizing different wavelengths of light. In this manner, the pulse oximetry sensor may provide physiological parameters related to respiratory and circulatory systems of the individual.

In certain cases, the pulse oximetry sensor may include an adhesive to enable application (e.g., adherence, attachment) to skin of the individual. After the application to the skin, the pulse oximetry sensor may emit light through the skin to measure the oxygen saturation levels in the blood of the individual.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it may be understood that these statements are to be read in this light, and not as admissions of prior art.

### SUMMARY

Certain embodiments commensurate in scope with the originally claimed subject matter are summarized below. These embodiments are not intended to limit the scope of the disclosure. Indeed, the present disclosure may encompass a variety of forms that may be similar to or different from the embodiments set forth below.

In certain embodiments, a medical sensor includes an outer cover that forms a pocket with an opening. The medical sensor also includes one or more electrical components positioned within the outer cover. The medical sensor further includes a biasing member coupled to the outer cover, wherein the biasing member adjusts from an initial shape to an adjusted shape to adjust a dimension of the opening to enable the opening to receive a portion of a patient.

In certain embodiments, a medical sensor includes an outer cover to surround a portion of a patient. The medical sensor also includes a flexible circuit with a light emitter and a light detector positioned within the outer cover. The medical sensor further includes a biasing member that adjusts from an initial shape to an adjusted shape to open the outer cover to receive the portion of the patient and adjusts from the adjusted shape to the initial shape to compress the outer cover about the portion of the patient.

Various refinements of the features noted above may exist in relation to various aspects of the present disclosure. Further features may also be incorporated in these various aspects as well. These refinements and additional features may exist individually or in any combination. For instance, various features discussed below in relation to one or more of the illustrated embodiments may be incorporated into any of the above-described aspects of the present disclosure alone or in any combination. The brief summary presented above is intended only to familiarize the reader with certain aspects and context of embodiments of the present disclosure without limitation to the claimed subject matter.

### BRIEF DESCRIPTION OF THE DRAWINGS

Advantages of the disclosed techniques may become apparent upon reading the following detailed description and upon reference to the drawings in which:

FIG. 1 is a perspective view of a medical monitoring system, in accordance with an embodiment of the present disclosure;

FIG. 2 is a block diagram of the medical monitoring system of FIG. 1, in accordance with an embodiment of the present disclosure;

FIG. 3 is a perspective exploded view of a sensor that may be employed in the medical monitoring system of FIG. 1, in accordance with an embodiment of the present disclosure;

FIG. 4 is a perspective view of the sensor of FIG. 3, wherein an outer cover is shown as transparent to facilitate visualization of other components of the sensor, in accordance with an embodiment of the present disclosure;

FIG. 5 is a perspective view of the sensor of FIG. 3 in an open configuration to facilitate removal of a liner, in accordance with an embodiment of the present disclosure;

FIG. 6 is a perspective view of the sensor of FIG. 3 in the open configuration to facilitate receiving a digit of a patient, in accordance with an embodiment of the present disclosure;

FIG. 7 is a perspective view of the sensor of FIG. 3 in the open configuration to receive the digit of the patient, in accordance with an embodiment of the present disclosure;

FIG. 8 is a perspective view of the sensor of FIG. 3 in a closed configuration to apply the sensor to the digit of the patient, in accordance with an embodiment of the present disclosure; and

FIG. 9 is a perspective view of the sensor of FIG. 3 with multiple biasing members, in accordance with an embodiment of the present disclosure.

### DETAILED DESCRIPTION OF THE INVENTION

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present disclosure, the articles “a,” “an,” and “the” are intended to mean that there are one or more of the elements. The terms “comprising,” “including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional embodiments that also incorporate the recited features.

It is presently recognized that it may be desirable to efficiently apply and reapply (e.g., reposition) a sensor on a patient for various reasons, such as to quickly place the sensor on the patient, adjust fit of the sensor on the patient, address skin irritation or discomfort of the patient due to the sensor, correct position of the sensor on the patient after movement of the patient, change position of the sensor on the patient to avoid interference with medical procedures, facilitate inspection of the sensor, and so forth. Additionally, it is presently recognized that it may be desirable to apply and reapply the sensor without reliance on adhesives to adhere the sensor to the patient, as such adhesives may cause discomfort for the patient and/or lose adhesive strength over multiple applications of the sensor to the patient. Further, it is presently recognized improved construction and design of the sensor may provide other advantages, such as light blocking, comfort, breathability, disposable components, and/or effective coupling to the patient even in presence of moisture (e.g., sweat on a portion of the patient at the sensor) and/or other factors that block adhesion between the patient and the sensor.

Accordingly, embodiments disclosed herein relate generally to a sensor that includes an outer cover (e.g., bandage; shroud) that surrounds one or more electrical components of the sensor. In certain embodiments, the one or more electrical components may include an emitter and a detector mounted to a flex circuit. An adhesive may be applied to a patient-facing side of the flex circuit (or to a patient-facing side of a metallized tape on the flex circuit) to facilitate coupling the one or more electrical components to the patient. Further, the sensor includes a biasing member (e.g., spring, such as an annular spring; expandable band; compliant structure) that surrounds and/or couples to the outer cover, and the biasing member may enable adjustment of the sensor between an open configuration to receive a digit of the patient and a closed configuration to apply the sensor to the digit of the patient. Thus, the sensor may be efficiently applied and reapplied (e.g., repositioned) on the patient. Additionally, while the adhesive may be present along the patient-facing side of the flex circuit (or the patient-facing side of the metallized tape on the flex circuit), the sensor utilizes the biasing member to provide sufficient force to apply and hold the sensor on the digit of the patient. Thus, the biasing member may support effective coupling of the sensor to the patient even in presence of moisture and/or other factors that block adhesion between the patient and the sensor. Additionally, the outer cover may provide light blocking, comfort, and breathability, and the sensor may be designed to be disposable after use by the patient.

While certain embodiments and examples provided herein describe the sensor as a pulse oximetry sensor, it should be appreciated that features may be utilized with any of a variety of sensor types. For example, the sensor may be a regional oximetry sensor, a bispectral index sensor, or other medical sensor. Indeed, as described herein, features of the sensor may be particularly useful for a medical sensor that is applied to a portion of a patient to monitor one or more physiological characteristics of the patient, especially in contexts or situations where it is desirable to firmly wrap the medical sensor around the portion of the patient, as well as to apply and reapply the medical sensor to the portion of the patient.

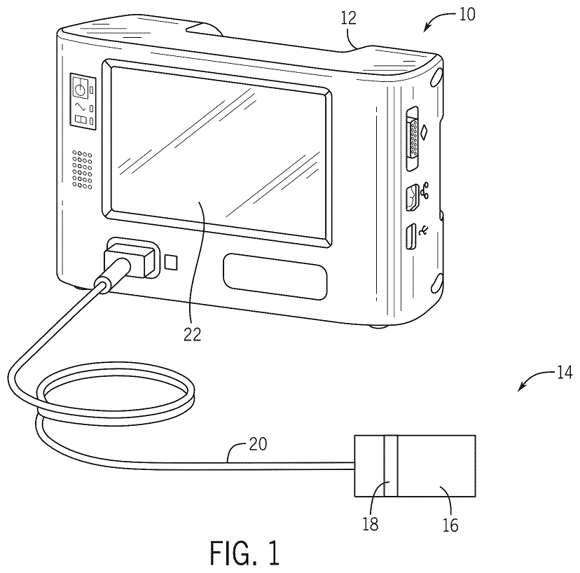

With the foregoing in mind, FIG. 1 is a perspective view of an embodiment of a medical monitoring system 10 that includes a monitor 12 (e.g., a patient monitor) that may be used in conjunction with a sensor 14 (e.g., a medical sensor). In the illustrated example, the monitor 12 is a pulse oximetry monitor and the sensor 14 is a pulse oximetry sensor. In such cases, the monitor 12 may be configured to process photoplethysmography (PPG) signals to calculate oxygen saturation (SpO2). It should be appreciated that the medical monitoring system 10 may be configured to obtain any of a variety of medical measurements and the techniques described herein may be adapted for use with any variety of monitors and sensors. For example, in some embodiments, the monitor 12 may include a regional oximeter and the sensor 14 may include a regional saturation sensor. In such cases, the monitor 12 may be configured to process the PPG signals to calculate regional oxygen saturation (rSO₂). Additionally, although the depicted embodiments illustrate the sensor 14 configured for use on a patient's finger, it should be understood that the sensor 14 may be adapted for use at other locations, such as an earlobe, forehead, toe, foot, ankle, wrist, or any other suitable measurement site (e.g., with pulsatile arterial flow).

As shown, the sensor 14 includes multiple layers or components, such as an outer cover 16 (e.g., bandage; shroud) and a biasing member 18 (e.g., spring, such as an annular spring; expandable band). As described in more detail herein, the sensor 14 may also include one or more electronic components, such as an emitter, a detector, and/or circuitry. In some embodiments, the one or more electronic components are coupled to or supported on a flex circuit (e.g., flexible circuit; flex circuit layer). When the multiple layers or components are assembled together, the biasing member 18 surrounds and/or couples to the outer cover 16, which surrounds or covers the one or more electronic components. In this way, the biasing member 18 compresses the outer cover 16 about a digit (e.g., finger) of the patient, and the outer cover 16 blocks ambient light from reaching the one or more electronic components (e.g., the detector). The sensor 14 may be reusable, disposable, partially usable, or partially disposable. However, it may be particularly desirable to form and design the sensor 14 to be disposable after use by the patient. To accomplish this, the sensor 14 may include few components (e.g., as compared to other sensors) and/or low cost components (e.g., as compared to other sensors). It should also be appreciated that the medical monitoring system 10 may include multiple sensors 14, such as multiple sensors 14 that are positioned on the patient at one time or over time and/or multiple sensors 14 that are positioned on multiple different patients at one time or over time.

As shown, the sensor 14 is communicatively coupled to the monitor 12. In the illustrated embodiment, the sensor 14 is coupled to the monitor 12 via a cable 20. The cable 20 may interface directly with the sensor 14 and may include multiple conductors (e.g., wires) to provide power, transmit signals, and/or receive signals. Additionally or alternatively, the sensor 14 may communicate with the monitor 12 wirelessly (e.g., the sensor 14 and the monitor 12 include wireless transceivers configured to communicate via any suitable wireless protocol). For example, the sensor 14 may include a transceiver that enables wireless signals to be transmitted to and/or received from an external device (e.g., the monitor 12). Additionally, the multiple conductors or the transceiver may transmit a raw digitized detector signal, a processed digitized detector signal, or a calculated physiological parameter, as well as any data that may be stored in the sensor 14. In operation, the monitor 12 may receive a signal from the sensor 14, and the monitor 12 may be configured to calculate or measure one or more physiological parameters based on the signal. In particular, the monitor 12 may include a processor configured to execute code (e.g., stored in a memory of the monitor 12 or received from another device) for filtering and processing the signal from the sensor 14 to calculate one or more physiological parameters, such as oxygen saturation. The monitor 12 may additionally or alternatively calculate any of a variety of physiological parameters, such as arterial blood oxygen saturation, regional or tissue oxygen saturation, pulse rate, respiration rate, blood pressure, blood pressure characteristic measure, autoregulation status, brain activity, or any other suitable physiological parameter.

Additionally, as illustrated in FIG. 1, the monitor 12 includes a display 22 configured to display one or more calculated physiological parameters, such as oxygen saturation. The display 22 may also display other information, such as instructions to charge the sensor 14, alarm indications, settings, and so forth. In certain embodiments, the display 22 may be a touch screen display. The monitor 12 may include various input components, such as the touch screen display, knobs, switches, keys and keypads, buttons, and so forth, to provide for operation and configuration of the monitor 12. The monitor 12 may also include one or more indicator lights and one or more speakers. The monitor 12 may also include additional slot(s) or wireless interfaces (e.g., channels) to connect to additional devices, such as additional sensors to monitor additional physiological parameters of the patient and/or to monitor physiological parameters of other patients at one time.

Furthermore, one or more functions of the monitor 12 disclosed herein may also be implemented directly in the sensor 14, or by any other suitable device. For example, in some embodiments, the sensor 14 may include one or more processing components configured to calculate one or more physiological parameters, such as oxygen saturation. The sensor 14 may have varying levels of processing power and may output data in various stages to the monitor 12. For example, in some embodiments, the data output to the monitor 12 may be analog signals, such as detected light signals (e.g., pulse oximetry signals or regional saturation signals), or processed data.

Further, in some embodiments, the sensor 14 may include a battery to provide power to components of the sensor 14. In some embodiments, the battery may be a rechargeable battery such as, for example, a lithium ion, a lithium polymer, a nickel-metal hydride, a nickel-cadmium battery, or any other suitable rechargeable battery. In other embodiments, any suitable power source may be utilized, such as, one or more capacitors or an energy harvesting power supply (e.g., a motion generated energy harvesting device, thermoelectric generated energy harvesting device, or any other suitable energy harvesting power supply). As described herein, the sensor 14 includes various structural features to provide comfort, breathability, and repositionability, as well as to block light (e.g., ambient light). Further, the sensor 14 may provide effective coupling to the patient even in presence of moisture and/or other factors that block adhesion between the patient and the sensor 14.

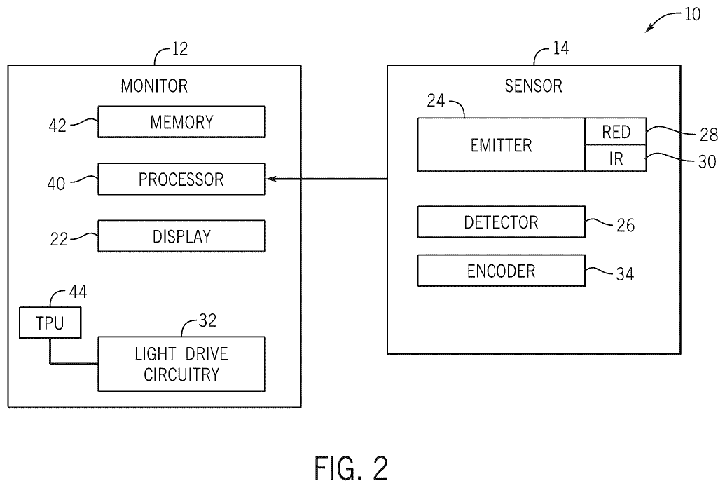

FIG. 2 illustrates a block diagram of an embodiment of the medical monitoring system 10. The sensor 14 includes optical components in the form of one or more emitters 24 (e.g., one or more light emitters; collectively referred to herein as an emitter for convenience) and one or more detectors 26 (e.g., one or more light detectors; collectively referred to herein as a detector for convenience). As shown, the emitter 24 includes two light emitting diodes (LEDs) that are configured to emit at least two wavelengths of light, e.g., a red LED 28 configured to emit wavelengths of light within the red spectrum and an infrared (IR) LED 30 configured to emit wavelengths of light within the infrared or near infrared spectrum. In one embodiment, the LEDs 28, 30 emit light in a range of about 600 nanometers (nm) to about 1000 nm. In one embodiment, the red LED 28 is configured to emit light between approximately 600 nm and 735 nm, and the IR LED 30 is configured to emit light between approximately 800 nm and 1000 nm. It should be noted that the emitter 24 may also transmit 3, 4, or 5 or more wavelengths of light in any suitable application.

As discussed in more detail herein, a light drive circuitry 32 of the monitor 12 may provide respective drive currents to the LEDs 28, 30 to cause the LEDs 28, 30 to emit respective wavelengths of light. It should be understood that, as used herein, the term “light” may refer to one or more of ultrasound, radio, microwave, millimeter wave, infrared, visible, ultraviolet, gamma ray or X-ray electromagnetic radiation, and may also include any wavelength within the radio, microwave, infrared, visible, ultraviolet, or X-ray spectra, and that any suitable wavelength of light may be appropriate for use with the present disclosure.

The emitter 24 emits light that passes through blood perfused tissue, and the detector 26 detects the light as reflected or transmitted by the tissue. The emitter 24 and the detector 26 may be arranged in a transmission configuration or a reflectance configuration with respect to one another. In the transmission configuration, the light enters the detector 26 after passing through the tissue of the patient. In the reflectance configuration, the light is reflected by elements in the tissue of the patient to enter the detector 26. In any case, the detector 26 may generate a signal (e.g., PPG signal) indicative of an intensity of the light received at the detector 26, and the detector 26 may send the signal to the monitor 12.

A signal representing light intensity versus time or a mathematical manipulation of this signal (e.g., a scaled version thereof, a log taken thereof, a scaled version of a log taken thereof) may be referred to as the PPG signal. Additionally, the term “PPG signal,” as used herein, may also refer to an absorption signal (e.g., representing an amount of light absorbed by the tissue) or any suitable mathematical manipulation thereof. The amount of light detected or absorbed may then be used to calculate any of a number of physiological parameters, including oxygen saturation (e.g., the saturation of oxygen in pulsatile blood, SpO2), an amount of a blood constituent (e.g., oxyhemoglobin), and/or a physiological rate (e.g., pulse rate or respiration rate; when each individual pulse or breath occurs). For SpO2, red and infrared (IR) wavelengths may be used because it has been observed that highly oxygenated blood will absorb relatively less red light and more IR light than blood with a lower oxygen saturation. By comparing the intensities of two wavelengths at different points in the pulse cycle, it is possible to estimate the blood oxygen saturation of hemoglobin in arterial blood, such as from empirical data that may be indexed by values of a ratio, a lookup table, from curve fitting, or other interpolative techniques.

As shown, the sensor 14 also includes an encoder 34. The encoder 34 may store information about the sensor 14, such as a type of sensor, calibration information, and so forth. When accessed by the monitor 12, the information about the sensor 14 may enable the monitor 12 to calculate oxygen saturation and/or other physiological parameters using the signal received from the detector 26. In certain embodiments, the sensor 14 may include sensing components in addition to, or instead of, the emitter 24 and the detector 26. For example, in one embodiment, the sensor 14 may include one or more actively powered electrodes (e.g., four electrodes) to obtain an electroencephalography signal.

As shown, the monitor 12 includes one or more processors 40, a memory 42, and the display 22. The processor 40 may process the signal received from the detector 26, such as by performing synchronized demodulation, amplification, and filtering of the signal. The processor 40 may process the signal received from the detector 26 to calculate one or more physiological parameters, such as the oxygen saturation, using various algorithms. Coefficients utilized in the algorithms may be accessed by the processor 40 from the encoder 34 or determined by the processor 40 based at on the calibration information of the sensor 14, for example. As noted herein, it should be appreciated that one or more functions or components of the monitor 12 disclosed herein may also be implemented directly in the sensor 14, or by any other suitable device. As described herein, the sensor 14 includes various structural features to provide comfort, breathability, and repositionability, as well as to block light. Further, the sensor 14 may provide effective coupling to the patient even in presence of moisture and/or other factors that block adhesion between the patient and the sensor,

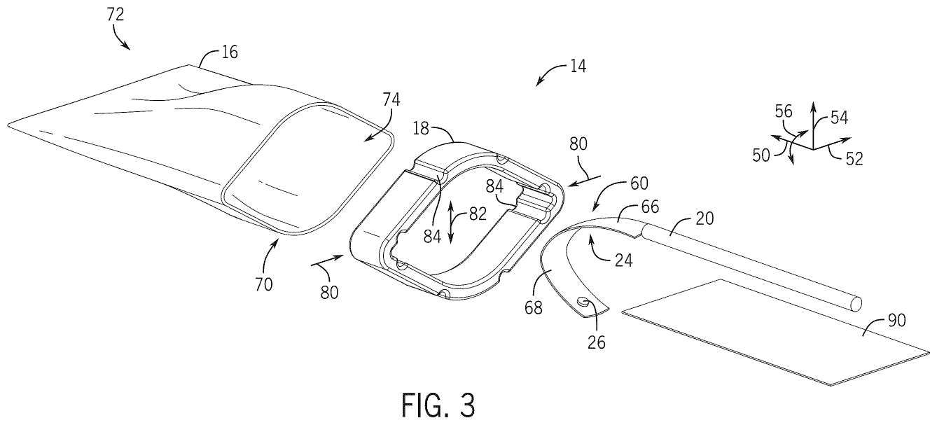

FIG. 3 is a perspective exploded view of an embodiment of the sensor 14 that may be employed in the medical monitoring system 10 of FIG. 1. To facilitate discussion, the sensor 14 and its components may be described with reference to a longitudinal axis or direction 50, a lateral axis or direction 52, a vertical axis or direction 54, and/or a circumferential axis or direction 56. It should be appreciated that the axes and directions are not intended to describe absolute directions (e.g., relative to gravity; relative to the patient), but instead are provided to facilitate discussion of relative positions of portions and features of the sensor 14.

As shown, the sensor 14 includes the outer cover 16 and the biasing member 18. The sensor 14 also includes a flex circuit 60 (e.g., flexible circuit; flex circuit layer) that supports optical components, such as the emitter 24 and the detector 26. The flex circuit 60 also supports circuitry (e.g., electrical lines) that extends from the emitter 24 and the detector 26 to the cable 20. In certain embodiments, a metallized tape 66 overlays or covers the flex circuit 60 to block ambient light. The metallized tape 66 may include an upper layer (e.g., outer layer) that overlays or covers an upper surface (e.g., outer surface; not a patient-facing surface) of the flex circuit 60, such as to overlay or cover an entirety of the upper surface of the flex circuit 60. Additionally, the metallized tape 66 may include a lower layer (e.g., inner layer) that overlays or covers a lower surface (e.g., inner surface; patient-facing surface) of the flex circuit 60, such as to overlay or cover the circuitry along portions of the lower surface of the flex circuit 60 while exposing the emitter 24 and the detector 26 (e.g., through openings in the metallized tape 66, wherein the openings are aligned with the emitter 24 and the detector 26). In this way, the metallized tape 66 surrounds the flex circuit 60 while exposing the emitter 24 and the detector 26 to enable transmission of light by the emitter 24 into tissue of the patient and detection of the light at the detector 26 after the light passes through the tissue of the patient.

Further, in certain embodiments, an adhesive 68 is applied or present along a patient-facing surface of the lower layer of the metallized tape 66 to facilitate adherence of this portion of the sensor 14 (e.g., including the emitter 24 and the detector 26) to the patient. The adhesive 68 may be a silicone-based adhesive, such as a silicone gel, and may minimize disruption of the skin of the patient (e.g., by removing only minimal skin protein) during removal of the sensor 14 from the patient. It should be appreciated that, in cases without the lower layer of the metallized tape 66, the adhesive 68 may be applied to a patient-facing surface of the flex circuit 60 or other layer that contacts the skin of the patient.

As shown, the flex circuit 60 with the metallized tape 66 and the adhesive 68 may bend, such as to position the emitter 24 and the detector 26 to face one another (e.g., along the vertical axis 54). Thus, the digit of the patient may be inserted or received between the emitter 24 and the detector 26, such that the emitter 24 and the detector 26 are positioned on opposite sides of the digit of the patient to facilitate monitoring one or more physiological characteristics of the patient, such as oxygen saturation. It should be appreciated that, in certain embodiments, the sensor 14 may be devoid of the adhesive 68. For example, in such cases, no adhesive is applied or present along the patient-facing surface of the lower layer of the metallized tape 66. Indeed, in some such cases, no adhesive is applied or present along any patient-contacting surface of the sensor 14, such that no adhesive contacts the patient while the sensor 14 is positioned on the patient for monitoring the patient.

Additionally, as shown, the outer cover 16 is designed to provide comfort to the patient, cover or protect the one or more electrical components (e.g., the flex circuit 60, the emitter 24, and the detector 26), and to block ambient light. Accordingly, the outer cover 16 may be formed from a flexible material (e.g., plastic, such polyethylene or polypropylene; fabric, such as with or without a lining or film) and/or may be fully or partially opaque to some or all wavelengths of light (e.g., visible light, IR light, and/or red light).

As shown, the outer cover 16 extends from a first end 70 (e.g., proximal end; end portion) to a second end 72 (e.g., distal end; end portion) along the longitudinal axis 50. The outer cover 16 is open at the first end 70 and closed at the second end 72. The outer cover 16 is closed along lateral edges and/or in the circumferential direction 56 between the first end 70 and the second end 72. In such cases, the outer cover 16 is only open at the first end 70 via an opening 74, while all other edges and ends are closed. In this way, the outer cover 16 may form a pocket (e.g., with a single opening, such as the opening 74) that covers the one or more electrical components when the sensor 14 is assembled and that also receives the digit of the patient through the opening 74 to facilitate the monitoring of the one or more physiological characteristics of the patient, such as oxygen saturation.

Further, as shown, the biasing member 18 is an annular structure that is sized to correspond to the opening 74 of the outer cover 16. For example, the biasing member 18 may be sized to couple to the outer cover 16, such as proximate to the opening 74 of the outer cover 16. As another example, the biasing member 18 may be sized to surround the outer cover 16, such as to wrap circumferentially around the outer cover 16.

In any case, the biasing member 18 may be a flexible member or spring that enables adjustment of the sensor 14 between an open configuration to receive the digit of the patient through the opening 74 and a closed configuration to apply the sensor 14 to the digit of the patient. The biasing member 18 may be formed (e.g., molded) to have an initial shape, such as a vertically compressed shape. The biasing member 18 may be flexible to assume or take an adjusted shape, such as a vertically expanded shape. Application of pressure (e.g., squeezing; compressing) to certain portions (e.g., opposed portions or sides) of the biasing member 18 may cause the biasing member 18 to transition from the initial shape to the adjusted shape. For example, application of pressure to compress opposed lateral sides of the biasing member 18 toward one another as shown by arrows 80 may cause the biasing member 18 to expand vertically as shown by arrows 82 to transition from the initial shape to the adjusted shape. When the biasing member 18 is in the initial shape, the biasing member 18 may cause the sensor 14 to be in the closed configuration. When the biasing member 18 is in the adjusted shape, the biasing member 18 may cause the sensor 14 to be in the open configuration.

As shown, the biasing member 18 may include structural features 84, such as grooves, at one or more locations of the biasing member 18. The structural features 84 may facilitate or promote bending or flexing of the biasing member 18 in certain desirable ways, such as to facilitate compression and expansion in certain dimensions that are suitable to allow insertion of the digit of the patient through the opening 74 and to provide application of the sensor 14 to the digit of the patient. Further, the structural features 84 may enable bending or flexing of the biasing member 18 in a manner that allows the sensor 14 to be used with a variety of patients (e.g., receiving and applying appropriate force to couple to digits of different dimensions).

As shown in FIG. 3, the structural features 84 include multiple grooves that extend along the longitudinal axis 50 (e.g., respective center axes extend along the longitudinal axis 50). For example, the structural features 84 may include the multiple grooves formed in an inner surface of the biasing member 18 and at opposed lateral sides of the biasing member 18. Additionally or alternatively, the structural features 84 may include the multiple grooves formed in an outer surface of the biasing member 18 and at opposed vertical sides of the biasing member 18.

More particularly, as shown in FIG. 3, the multiple grooves formed in the inner surface of the biasing member 18 and at opposed lateral sides of the biasing member 18 may include a first set of multiple grooves (e.g., three grooves) formed in the inner surface of the biasing member 18 and at a first lateral side of the biasing member 18, as well as a second set of multiple grooves (e.g., three grooves) formed in the inner surface of the biasing member 18 and at a second lateral side of the biasing member 18. Further, as shown in FIG. 3, the multiple grooves formed in the outer surface of the biasing member 18 and at opposed vertical sides of the biasing member 18 may include a third set of multiple grooves (e.g., two grooves) spaced apart along the lateral axis 52 and formed in the outer surface of the biasing member 18 on a first vertical side of the biasing member 18. Further, the multiple grooves formed in the outer surface of the biasing member 18 and at opposed vertical sides of the biasing member 18 may include a fourth set of multiple grooves (e.g., two grooves) spaced apart along the lateral axis 52 and formed in the outer surface of the biasing member 18 on a second vertical side of the biasing member 18.

In any case, the multiple grooves formed in the biasing member 18 may facilitate vertical expansion of the biasing member 18, as shown by the arrows 82, upon application of the compression to the opposed lateral sides of the biasing member 18, as shown by the arrows 80. Further, the multiple grooves formed in the biasing member 18 may also facilitate return to the initial shape upon release of the compression to the opposed lateral sides of the biasing member 18. Additional details related to structural and operational features of the biasing member 18 are described in more detail herein.

In certain embodiments, a liner 90 may be positioned between the emitter 24 and the detector 26 during manufacture of the sensor 14 and prior to use of the sensor 14 by the patient. In particular, the liner 90 may be positioned between the emitter 24 and the detector 26 and within the outer cover 16, and the liner 90 may include a coating to preserve the adhesive 68 and/or block adhesion of different portions of the adhesive 68 to one another or the rest of the sensor 14.

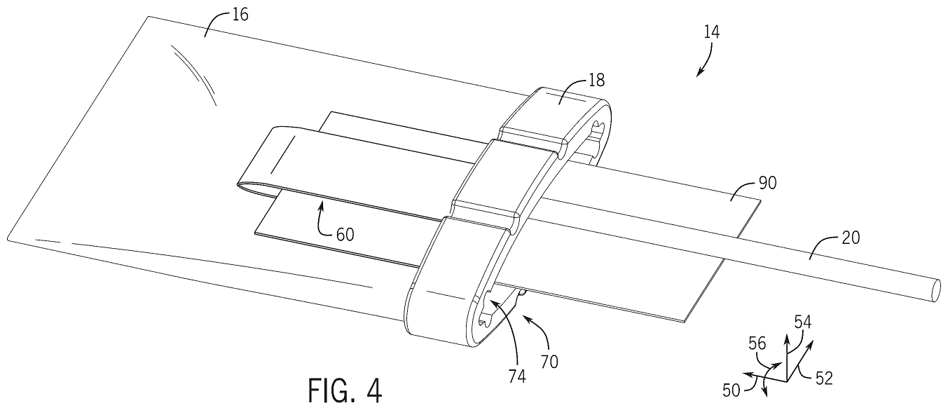

FIG. 4 is a perspective view of an embodiment of the sensor 14, wherein the outer cover 16 is shown as transparent to facilitate illustration of other components of the sensor 14. In FIG. 4, the sensor 14 is assembled, such that the outer cover 16 surrounds the flex circuit 60 and such that the biasing member 18 surrounds and/or couples to the outer cover 16. In particular, the outer cover 16 covers the flex circuit 60 to thereby protect the one or more electrical components of the sensor 14, as well as to block ambient light from reaching the detector of the sensor 14 (e.g., presence of ambient light may interfere with or result in inaccurate measurements of the one or more physiological characteristics of the patient). Further, the biasing member 18 is positioned at or proximate to the first end 70 of the outer cover 16 to thereby control or adjust dimensions (e.g., a size and/or a shape) of the opening 74. For example, compression of certain portions of the biasing member 18 may cause the biasing member 18 to transition from the initial shape to the adjusted shape, which may in turn adjust the dimensions of the opening 74. In particular, upon the compression of the certain portions of the biasing member 18, the biasing member 18 applies forces to the outer cover 16 and causes the dimensions of the opening 74 to generally match or correspond to the adjusted shape of the biasing member 18.

In certain embodiments, the biasing member 18 is coupled to the outer cover 16, such as via adhesives, fasteners, or welds. In certain embodiments, the biasing member is coupled to the outer cover 16 by being integrally formed with the outer cover 16, such as via molding techniques (e.g., to form a one-piece structure). In any case, with the biasing member 18 coupled to the outer cover 16, the biasing member 18 is designed and expected to remain attached or fixed to the outer cover 16 during a patient monitoring session (e.g., the biasing member 18 is not to be removed by an operator, such as the patient or a medical professional; separation of the biasing member 18 from the outer cover 16 may damage the sensor 14). Further, in such cases, the dimensions of the opening 74 generally match or correspond to the adjusted shape of the biasing member 18 (e.g., the first end 70 of the outer cover 16 that defines the opening 74 moves with the biasing member 18).

It should be appreciated that, in certain embodiments, the biasing member 18 is a separate piece that may be separately applied and removed from the outer cover 16 during a patient monitoring session (e.g., the biasing member 18 may be removed by the operator as part of recommended or acceptable procedures for use of the sensor 14). Thus, in such cases, the biasing member 18 is not fixed to the outer cover 16, but instead the biasing member 18 is temporarily positioned about the outer cover 16 to contact and apply forces to the outer cover 16 during the patient monitoring session.

In certain embodiments, the flex circuit 60 is coupled to the outer cover 16, such as via adhesives, fasteners, or welds. In certain embodiments, the cable 20 is coupled to the outer cover 16 and/or the biasing member 18, such as via adhesives, fasteners, or welds. As shown in FIG. 4, the flex circuit 60 bends to position the emitter and the detector to face one another (e.g., along the vertical axis 54). Further, the liner 90 is inserted between the emitter and detector to position a desired surfaces (e.g., coated surfaces) of the liner 90 along the adhesive 68. It should be appreciated that the liner 90 may also bend to position one desired surface (e.g., one coated surface) of the liner 90 along the adhesive 68.

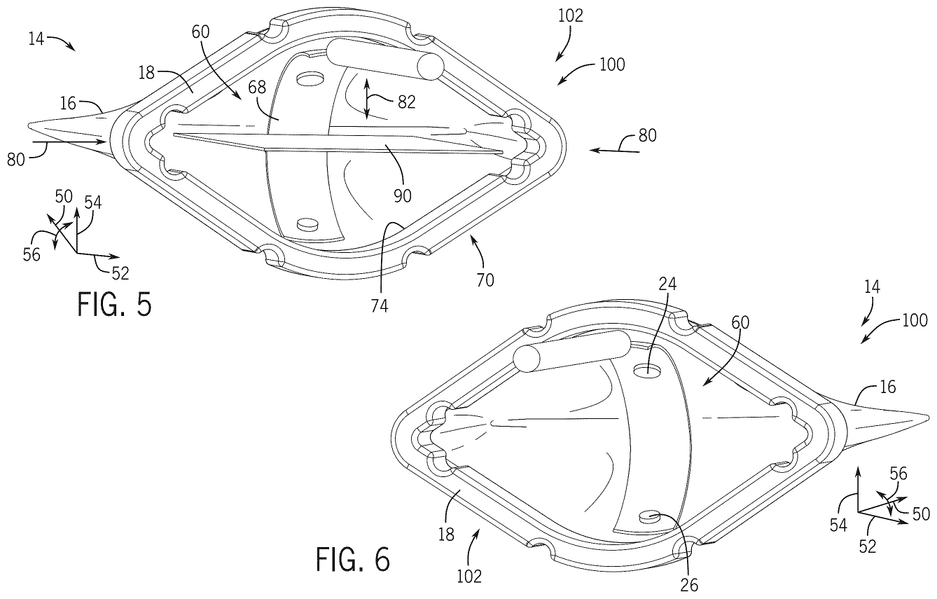

FIG. 5 is a perspective view of an embodiment of the sensor 14 in an open configuration 100 to facilitate removal of the liner 90. In the open configuration 100, the opening 74 defined at the first end 70 of the outer cover 16 has dimensions (e.g., a size and/or a shape) that enable access to the liner 90 for removal of the liner 90. For example, the operator may grip the liner 90 and apply force to pull the liner 90 through the opening 74 and out of the outer cover 16.

In FIG. 5, the biasing member 18 is in an adjusted shape 102 (e.g., adjusted configuration). As described herein, the transition of the biasing member 18 from the initial shape to the adjusted shape 102 may cause the sensor 14 to transition from the closed configuration to the open configuration 100. For example, the operator may compress the opposed lateral sides of the biasing member 18 toward one another as shown by the arrows 80, which may cause the biasing member 18 to expand vertically as shown by the arrows 82 to transition the biasing member 18 from the initial shape to the adjusted shape 102. As the biasing member 18 expands vertically as shown by the arrows 82, the outer cover 16, the opening 74 defined by the outer cover 16, and the flex circuit 60 coupled to the outer cover 16 may also expand vertically as shown by the arrows 82. Accordingly, the flex circuit 60 (e.g., the adhesive 68) may separate from the liner 90, which may facilitate the removal of the liner 90.

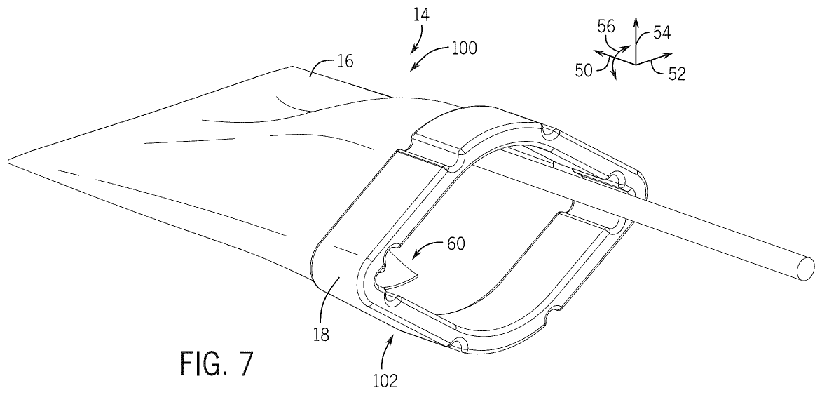

FIG. 6 and FIG. 7 are perspective views of an embodiment of the sensor 14 in the open configuration 100 to facilitate receiving the digit of the patient. As shown, the biasing member 18 is in the adjusted shape 102 to cause the sensor 14 to be in the open configuration 100 to facilitate receiving the digit of the patient. Further, the emitter 24 and the detector 26 of the flex circuit 60 are positioned to receive the digit of the patient between the emitter 24 and the detector 26 (e.g., along the vertical axis 54).

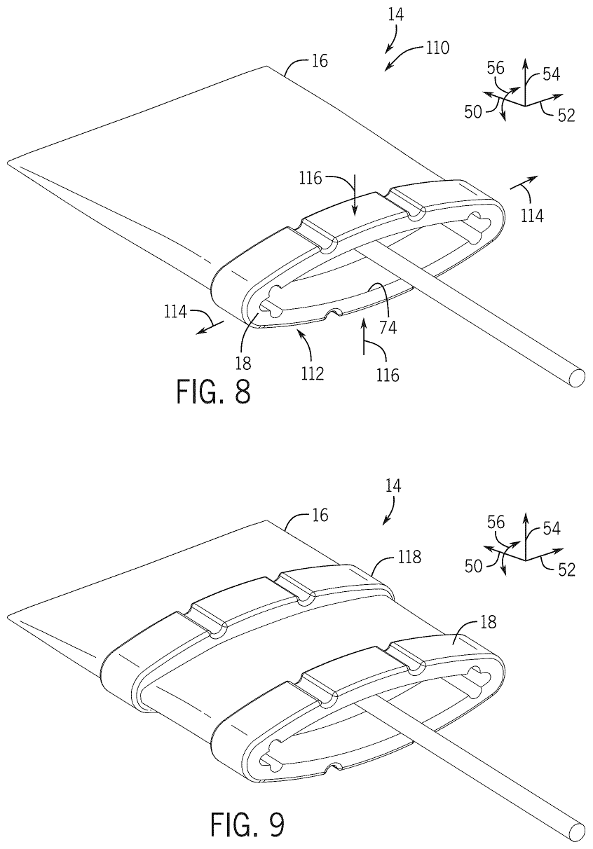

FIG. 8 is a perspective view of an embodiment of the sensor 14 in a closed configuration 110 to apply the sensor 14 to the digit of the patient. In the closed shape 102, the opening 74 defined at the first end 70 of the outer cover 16 has dimensions (e.g., a size and/or a shape) that compress the outer cover 16 about or onto the digit of the patient.

In FIG. 8, the biasing member 18 is in an initial shape 112 (e.g., initial configuration). As described herein, the transition of the biasing member 18 from the adjusted shape 102 of FIGS. 5-7 to the initial shape 112 of FIG. 8 may cause the sensor 14 to transition from the open configuration 100 of FIGS. 5-7 to the closed configuration 110 of FIG. 8. For example, the operator may release (e.g., remove the compression from) the opposed lateral sides of the biasing member 18, which may cause the biasing member 18 to expand laterally and compress vertically as shown by the arrows 114, 116, respectively, to transition the biasing member 18 from the adjusted shape 102 to the initial shape 112. As the biasing member 18 expands laterally and compresses vertically as shown by the arrows 114, 116, respectively, the outer cover 16, the opening 74 defined by the outer cover 16, and the flex circuit 60 coupled to the outer cover 16 may also at least compress vertically, as shown by the arrows 116.

Accordingly, the flex circuit 60 (e.g., the adhesive 68 of the flex circuit 60) may adhere to the patient and/or may position the emitter 24 and the detector 26 to contact the patient to facilitate the monitoring of the one or more physiological characteristics of the patient, such as oxygen saturation. Because the biasing member 18 has the initial shape 112 and is molded with the initial shape 112, the biasing member 18 is biased to move to and to maintain the initial shape 112 in absence of external forces or pressure. Thus, the biasing member 18 holds the sensor 14 in the closed configuration 110 during a patient monitoring session of the patient and/or as long as the external forces or pressure are not applied to the biasing member 18 (e.g., as long as the external forces or pressure are insufficient to adjust the biasing member 18 to the adjusted shape 102).

Advantageously, the sensor 14 may be quickly and efficiently placed on the patient, such as by following steps described and outlined with respect to FIGS. 5-8. Further, the sensor 14 may be quickly and efficiently removed from the patient and/or repositioned on the patient, such as by compressing the opposed lateral sides of the biasing member 18 toward one another, which may cause the biasing member 18 to expand vertically to transition the biasing member 18 from the initial shape 112 to the adjusted shape 102. As the biasing member 18 expands vertically, the outer cover 16, the opening 74 defined by the outer cover 16, and the flex circuit 60 coupled to the outer cover 16 may also expand vertically. Accordingly, the flex circuit 60 (e.g., the adhesive 68) may separate from the digit of the patient, which may facilitate the removal of the sensor 14 from the digit of the patient and/or repositioning of the sensor 14 on the digit of the patient. For example, with the biasing member 18 in the adjusted shape 102 and the sensor 14 in the open configuration 100, the operator may shift the sensor 14 relative to the digit of the patient to reposition the sensor 14 on the digit of the patient. As another example, with the biasing member 18 in the adjusted shape 102 and the sensor 14 in the open configuration 100, the operator may remove the sensor 14 from the digit of the patient and then reposition the sensor 14 on another digit of the patient.

It should be appreciated that once the sensor 14 is located in a desirable position (e.g., after repositioning), the operator may release (e.g., remove the compression from) the opposed lateral sides of the biasing member 18, which may cause the biasing member 18 to expand laterally and compress vertically to transition the biasing member 18 from the adjusted shape 102 to the initial shape 112. As the biasing member 18 expands laterally and compresses vertically, the outer cover 16, the opening 74 defined by the outer cover 16, and the flex circuit 60 coupled to the outer cover 16 may also at least compress vertically to apply the sensor 14 to the patient to facilitate the monitoring of the one or more physiological characteristics of the patient, such as oxygen saturation.

Accordingly, the sensor 14 described herein provides various advantages with respect to application, removal, and/or repositioning on the patient. Additionally, the sensor 14 provides other desirable features, such as light blocking and comfort for the patient. Further, the sensor 14 provides a low component count and associated low cost, and the sensor 14 may also be designed to be disposable after use by the patient.

FIG. 9 is a perspective view of an embodiment of the sensor 14 with an additional biasing member 118. In certain embodiments, it may be desirable to provide the additional biasing member 118 to surround and/or coupled to the outer cover 16 of the sensor 14. For example, the additional biasing member 118 may provide additional compression to apply the sensor 14 to the digit of the patient, which may assist with holding or maintaining the sensor 14 on the digit of the patient. The biasing member 18 and the additional biasing member 118 may be spaced apart from one another along the longitudinal axis 50, such that the biasing member 18 and the additional biasing member 118 circumferentially surround different portions of the outer cover 16 and compress the outer cover 16 against different portions of the digit of the patient.

In certain embodiments, the biasing member 18 and the additional biasing member 118 may be identical with a same size and a same shape (e.g., a same width along the longitudinal axis 50; a same initial shape, such as the initial shape 112). In certain embodiments, the biasing member 18 and the additional biasing member 118 may be different with respect to size and/or shape (e.g., different widths along the longitudinal axis 50; different initial shapes). In such cases, the different widths and/or the different initial shapes may assist in application of the sensor 14 to digits having different parameters, such as different sizes and/or shapes. It should be appreciated that the sensor 14 may include any number of biasing members, such as 1, 2, 3, 4, or more, and the biasing members may be distributed or spaced apart along the longitudinal axis 50. Further, with multiple biasing members, the multiple biasing members may include the same size and the same shape, or the multiple biasing members may be different with respect to size and/or shape. Further, with multiple biasing members, the multiple biasing members may transition from respective initial shapes to respective adjusted shapes together, such as via compression applied simultaneously to the multiple biasing members (and also transition from respective adjusted shapes to respective initial shapes together, such as via release of the compression simultaneously).

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Rather, the various embodiments may cover all modifications, equivalents, and alternatives falling within the spirit and scope of the disclosure as defined by the following appended claims. For example, the biasing member(s) may have any suitable width and/or be at any suitable location along the outer cover (e.g., relative to the longitudinal axis). Additionally, features described herein or shown in FIGS. 1-9 may be combined in any suitable manner.

What is claimed is:

1. A medical sensor, comprising:
an outer cover that forms a pocket with an opening;
one or more electrical components positioned within the outer cover; and
a biasing member coupled to the outer cover, wherein the biasing member adjusts from an initial shape to an adjusted shape to adjust a dimension of the opening to enable the opening to receive a portion of a patient.

2. The medical sensor of claim 1, wherein the outer cover comprises an opaque material to block at least some wavelengths of light.

3. The medical sensor of claim 1, wherein the one or more electrical components comprise a light emitter and a light detector.

4. The medical sensor of claim 3, wherein the light emitter and the light detector are supported on a flex circuit.

5. The medical sensor claim 4, wherein the flex circuit is coupled to the outer cover.

6. The medical sensor of claim 4, comprising a metallized tape coupled to the flex circuit.

7. The medical sensor of claim 6, comprising an adhesive disposed on a patient-facing surface of the metallized tape.

8. The medical sensor of claim 1, wherein the biasing member comprises an annular shape.

9. The medical sensor of claim 1, wherein the biasing member comprises a first vertical dimension in the initial shape, a second vertical dimension in the adjusted shape, and the first vertical dimension is greater than the second vertical dimension.

10. The medical sensor of claim 1, wherein the biasing member adjusts from the initial shape to the adjusted shape in response to compression at opposed portions of the biasing member.

11. The medical sensor of claim 10, wherein the biasing member adjusts from the adjusted shape to the initial shape to adjust the dimension of the opening to compress the outer cover about the portion of the patient, and the biasing member adjusts from the adjusted shape to the initial shape in response to release of the compression at the opposed portions of the biasing member.

12. A medical sensor, comprising:
an outer cover to surround a portion of a patient;
a flexible circuit with a light emitter and a light detector positioned within the outer cover; and
a biasing member that adjusts from an initial shape to an adjusted shape to open the outer cover to receive the portion of the patient and adjusts from the adjusted shape to the initial shape to compress the outer cover about the portion of the patient.

13. The medical sensor of claim 12, wherein the biasing member comprises an annular spring that is biased toward the initial shape.

14. The medical sensor of claim 12, wherein the biasing member is coupled to the outer cover proximate to an opening of the outer cover that receives the portion of the patient into the outer cover.

15. The medical sensor of claim 12, wherein the biasing member adjusts from the initial shape to the adjusted shape in response to compression at opposed portions of the biasing member, and the biasing member adjusts from the adjusted shape to the initial shape in response to release of the compression at the opposed portions of the biasing member.
